SEQUENCE LISTING (2 sequences)

SEQ ID NO: 1 - protein, 39 aa, Artificial Sequence
  Exendin-4
HGEGTFTSDLSKQMEEEAVRLFIEWLKNGGPSSGAPPPS

SEQ ID NO: 2 - protein, 40 aa, Artificial Sequence
  polypeptide
HGEGTFTSDLSKQMEEEAVRLFIEWLKNGGPSSGAPPPSC

(19) United States
(12) Patent Application Publication
(10) Pub. No.: US 2023/0048956 A1
(43) Pub. Date: Feb. 16, 2023

(54) BIOTIN MOIETY-CONJUGATED POLYPEPTIDE AND PHARMACEUTICAL COMPOSITION FOR ORAL ADMINISTRATION COMPRISING THE SAME

(71) Applicant: D&D Pharmatech Inc., Gyeonggi-do (KR)

(72) Inventors: Ok-cheol Jeon, Seoul (KR); Eun Ji Park, Seoul (KR)

(21) Appl. No.: 17/778,759

(22) PCT Filed: Nov. 20, 2020
(86) PCT No.: PCT/KR2020/016531

§ 371 Date: May 20, 2022

(30) Foreign Application Priority Data
Nov. 27, 2019 (KR) 10-2019-0153983

(51) Int. Cl. A61K 47/54; A61K 47/42; A61K 9/00; A61P 1/16; A61P 25/28

(52) U.S. Cl. CPC A61K 47/557; A61K 47/42; A61K 9/0053; A61P 1/16; A61P 25/28

(57) ABSTRACT

The present invention relates to a biotin moiety-conjugated polypeptide and a pharmaceutical composition for oral administration comprising the same, wherein the polypeptide according to the present invention has an excellent in vivo oral bioavailability.

### TECHNICAL FIELD

The present invention relates to a biotin moiety-conjugated polypeptide and a pharmaceutical composition for oral administration comprising the same, and more particularly, to a biotin moiety -conjugated polypeptide and a pharmaceutical composition for oral administration comprising the same having an excellent oral bioavailability without reducing the activity of polypeptides.

### BACKGROUND ART

Recently, through the rapid growth of scientific technology with the economic development, dietary habits have been changed, and the intake of high-calorie-high-fat foods has been increased. Accordingly, the population of adult diseases such as diabetes and obesity due to various metabolic diseases is rapidly increasing.

Diabetes causes a variety of complications and largely deteriorates the quality of life of patients due to a moderate diet, and thus, the awareness of treatment and management is increasing, and the development of a therapeutic agent for improving or treating diabetes is urgently needed.

Diabetes is divided into ‘Type I diabetes’, which is caused by a decrease in insulin secretion, and ‘Type II diabetes’, which is caused by a decrease in metabolic regulatory ability due to insulin resistance while insulin production is normal. Type II diabetes and obesity are mutual causes of disease and are very dangerous diseases due to an increased risk of atherosclerosis which is not only a cause of metabolic disease, but also a major cause of death in diabetic patients.

Glucagon-like peptide-1 (GLP-1) induces various biological effects, such as stimulation of insulin secretion, suppression of glucagon secretion, suppression of gastric emptying, suppression of gastric movement and bowel movement, promotion of glucagon use, and induction of weight loss. In addition, the GLP-1 is known to act to prevent pancreatic b-cell degeneration caused by progression of non-insulin dependence diabetes mellitus (NIDDM), which is Type II diabetes, and recover insulin secretion ability by promoting the production of b-cells. In particular, a remarkable characteristic of the GLP-1 is having an ability to stimulate insulin secretion without accompanying the risk associated with hypoglycemia shown in the case of using insulin therapy, or other drugs increasing insulin expression. In addition, since it is known that the GLP-1 does not accompany side effects such as death of b-cells in the pancreas and necrosis caused by long-term administration of a hypoglycemic agent, sulfonylurea or the like, it is very effective in the treatment of Type II diabetes.

It has been known that exendin-4, which is obtained from the saliva of Gila Monster Lizard with about 50% of amino acid homology with GLP-1, also activates a GLP-1 receptor to alleviate hyperglycemia in diabetic patients. In addition, it is known that the exendin-4 has effects such as stimulation of insulin secretion, suppression of glucagon secretion, suppression of gastric emptying, suppression of gastric movement and bowel movement, promotion of glucagon use, and induction of weight loss (U.S. Pat. No. 5,424,286). In addition, the exendin-4 is known to be effective in treating obesity (Pharmacol Rev 70:712-746; Anti-Obesity Therapy: from Rainbow Pills to Polyagonists). In addition, the exendin-4 is known to have an effect in treating non-alcoholic fatty liver disease ([1] World J Gastroenterol 2014 Oct. 28; 20(40): 14821-14830, [2] Am J Physiol Gastrointest Liver Physiol 302: G762- G772, 2012, [3] J Huazhong Univ Sci Technol [Med Sci] 35(3): 333-336, 2015, [4] The American Journal of Pathology, Vol. 181, No. 5, November 2012). In addition, the exendin-4 is known to have a therapeutic effect on neurodegenerative diseases, such as Alzheimer's disease (The Journal of clinical investigation 122:1339-1353;). In 2005, synthetic exendin-4 was commercially approved by the US Food and Drug Administration under the trademark Byetta™, and has been used for the treatment of Type II diabetes.

However, when peptide drugs such as exendin-4 are administered orally, there is a problem in that the peptide drugs are degraded due to a digestive enzyme and are not penetrated into the intestinal membrane, and thus, parenteral administration, such as subcutaneous administration, has been performed.

Therefore, there is a need for a technology that protects the degradation of peptides from enzymes and ultimately penetrates into the intestinal membrane through a carrier to improve the absorption in the intestine. As a related prior art, Korean Patent Registration No. 10-0864584 discloses that an exendin-4 derivative in which biotin is modified in a lysine residue of exendin-4 may be administered orally and the bioavailability in the intestine is improved. However, in this case, there is a problem in that biotin is conjugated to various lysine positions of exendin-4 to form various isomers, thereby lowering the reaction rate and yield, and biotin is conjugated to a lysine position of an N-terminal which is an active site of exendin-4 to reduce the activity of exendin-4.

Therefore, the present inventors have made efforts to develop peptide drugs that can be administered orally without reducing the activity of exendin-4 with a peptide, and as a result, have confirmed that a polypeptide in which a biotin moiety is selectively conjugated to cysteine is prepared by using a polypeptide in which the cysteine is inserted to an inactive site of exendin-4 to produce a uniform material without formation of isomers, thereby improving the reaction rate and yield. In addition, the present inventors have confirmed that the biotin moiety-conjugated polypeptide exhibited an excellent oral bioavailability without reducing the activity of exendin-4, resulting in improved glucose control ability. Therefore, the present inventors have found that the biotin moiety-conjugated polypeptide according to the present invention may be used as a pharmaceutical composition that can be administered orally and then completed the present invention.

### DISCLOSURE OF INVENTION

### Technical Problem

An object of the present invention is to provide a biotin moiety-conjugated polypeptide having an excellent in vivo oral bioavailability without reducing the activity of polypeptides, and a pharmaceutical composition for oral administration comprising the same.

### Solution to Problem

An aspect of the present invention provides a biotin moiety-conjugated polypeptide,

wherein the polypeptide is the one with insertions or substitutions of one or more amino acid residue between 9^{th} and 39^{th} in SEQ ID NO:1 with cysteine residue, and

a biotin moiety represented by the following General Formula A is conjugated to cysteine residue of the above polypeptide.

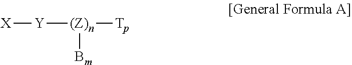

wherein,

X is a functional group capable of conjugation to the polypeptide,

Y is a spacer,

Z is a binding unit,

B may be represented by the following Chemical Formula A-1,

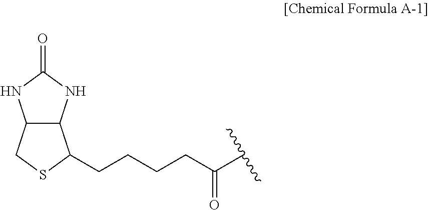 [SMILES: *C(=O)CCCCC1SCC2NC(=O)NC21]

T is a terminal group,

m is an integer of 1 to 10,

n is an integer of 0 or 1 to 10, and

p is an integer of 0 or 1.

Another aspect of the present invention provides a pharmaceutical composition for oral administration comprising the biotin moiety-conjugated polypeptide.

Yet another aspect of the present invention provides a pharmaceutical composition for preventing or treating obesity, diabetes, fatty liver disease, or neurodegenerative disease, comprising the biotin moiety-conjugated polypeptide.

Still another aspect of the present invention provides a method for preparing a biotin moiety-conjugated polypeptide comprising:

1) obtaining a polypeptide in which at least one of amino acids 9 to 39 of a polypeptide consisting of an amino acid sequence represented by SEQ ID NO: 1 is substituted or inserted with cysteine;

2) obtaining a polypeptide in which a biotin moiety is conjugated to cysteine by reacting the polypeptide obtained in step 1) and the biotin moiety represented by the following General Formula A in an organic solvent; and

3) isolating and purifying the biotin moiety-conjugated polypeptide of step 2);

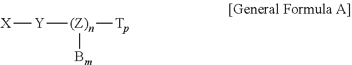

wherein,

X is a functional group capable of conjugation to the polypeptide,

Y is a spacer,

Z is a binding unit,

B may be represented by the following Chemical Formula A-1,

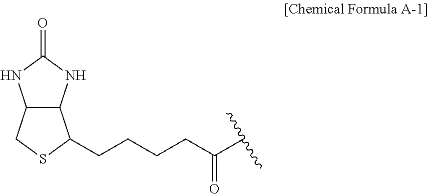 [SMILES: *C(=O)CCCCC1SCC2NC(=O)NC21]

T is a terminal group,

m is an integer of 1 to 10,

n is an integer of 0 or 1 to 10, and

p is an integer of 0 or 1.

### ADVANTAGEOUS EFFECTS OF INVENTION

According to an embodiment of the present invention, by preparing a polypeptide with cysteine inserted at the C-terminus which is an inactive site of exendin-4, and a polypeptide in which a biotin moiety is selectively bound to cysteine according to the present invention using a biotin moiety, it is possible to produces a uniform material without forming isomers, and has increased reaction rate and yield. In addition, it is possible to selectively bind the biotin moiety to cysteine without affecting the biological activity, thus not inhibiting the biological activity of the polypeptide.

The biotin moiety-conjugated polypeptide according to an embodiment of the present invention may have excellent oral bioavailability.

The biotin moiety-conjugated polypeptide according to an embodiment of the present invention may protect the polypeptide from being degraded from enzymes, and ultimately penetrate the intestinal membrane through a receptor in the body to promote bioavailability in the intestine.

The biotin moiety-conjugated polypeptide according to an embodiment of the present invention may be absorbed active transport through a sodium-dependent multivitamin transporter by binding with biotin, which is a type of water-soluble vitamins B7.

The biotin moiety-conjugated polypeptide according to an embodiment of the present invention may be used as a composition for oral administration, and specifically, may be used as a composition for oral administration for preventing or treating diabetes, obesity, fatty liver disease, or neurodegenerative disease.

### BRIEF DESCRIPTION OF DRAWINGS

FIG. 1 is a purification chromatogram of Example 3 according to an embodiment of the present invention;

FIG. 2 is HPLC chromatograms of Examples 1 to 3 according to an embodiment of the present invention;

FIG. 3 is MALDI-TOF mass spectra of Examples 1 to 3 according to an embodiment of the present invention;

FIG. 4 is a schematic diagram illustrating cleavage after an endoproteinase Lys-C treatment to identify the biotin moiety conjugated site of Example 2 and 3 according to an embodiment of the present invention;

FIG. 5 is HPLC chromatograms after enzymatic digestion using an endoproteinase Lys-C of Example 2 and 3 compared to SEQ ID NO:2 according to an embodiment of the present invention;

FIG. 6 is MALDI-TOF mass spectra of each fragment produced after enzymatic digestion using an endoproteinase Lys-C of Example 2 and 3 according to an embodiment of the present invention;

FIG. 7 is a graph showing biological activities of Examples 1 to 3 compared to SEQ ID NO:2 according to an embodiment of the present invention;

FIG. 8 is a graph showing biological activities of Example 3 and Comparative Example 1 compared to SEQ ID NO:2 according to an embodiment of the present invention;

FIG. 9 is a graph showing blood concentrations over time after oral administration to rats of Examples 1 to 3 according to an embodiment of the present invention; and

FIG. 10 is a graph showing changes in blood glucose levels over time after intraperitoneal glucose tolerance test of Examples 1 to 3 according to an embodiment of the present invention.

### BEST MODE FOR CARRYING OUT THE INVENTION

The biotin moiety-conjugated polypeptide, wherein the biotin moiety is represented by any one of the following Formulas;

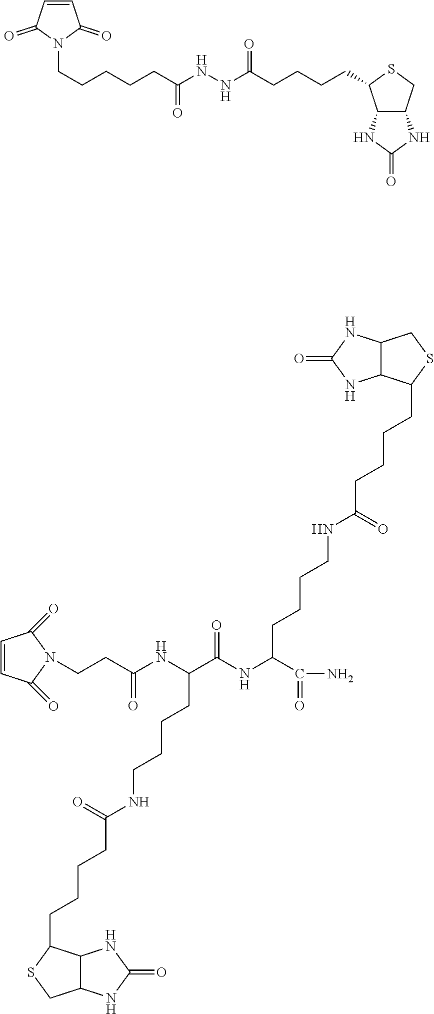 [SMILES: O=C(CCCCCN1C(=O)C=CC1=O)NNC(=O)CCCC[C@@H]1SC[C@@H]2NC(=O)N[C@@H]21 ; NC(=O)C(CCCCNC(=O)CCCCC1SCC2NC(=O)NC21)NC(=O)C(CCCCNC(=O)CCCCC1SCC2NC(=O)NC21)NC(=O)CCN1C(=O)C=CC1=O]

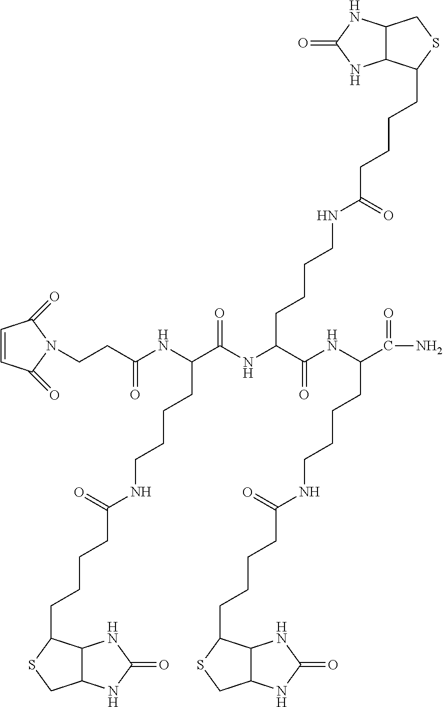 [SMILES: NC(=O)C(CCCCNC(=O)CCCCC1SCC2NC(=O)NC21)NC(=O)C(CCCCNC(=O)CCCCC1SCC2NC(=O)NC21)NC(=O)C(CCCCNC(=O)CCCCC1SCC2NC(=O)NC21)NC(=O)CCN1C(=O)C=CC1=O]

### MODE FOR THE INVENTION

Hereinafter, embodiments and Examples of the present invention will be described in detail such that those skilled in the art to which the present invention pertains can easily implement the present invention.

However, the present invention may be embodied in many different forms and are not limited to embodiments and Examples described herein. Throughout this specification, unless otherwise particularly described to the contrary, when any part “comprises” any component, it may be meant that another component may be further included without excluding another component.

The terms “about”, “substantially”, and the like indicating a degree to be used in this specification are used as a numerical value or a value close to the numerical value when inherent manufacturing and material tolerances are presented in the stated meaning, and used to prevent an unscrupulous infringer from unfairly using disclosed contents in which precise or absolute numerical values are mentioned to help in the understanding of the present invention. In the specification, the term “step (of˜)” or “step of” of a degree to be used in this specification does not mean “step for”.

Throughout this specification, the term “combination thereof” included in the expression of the Markush form means mixture or combination of at least one selected from the group consisting of components described in the expression of the Markush form, and means including at least one selected from the group consisting of the components. Throughout the present specification, “A and/or B” means “A and B, or A or B”.

An aspect of the present invention provides a polypeptide in which a biotin moiety is conjugated to cysteine. The biotin moiety-conjugated polypeptide according to an aspect of the present invention is conjugated with a water-soluble biotin moiety to have an excellent in vivo oral bioavailability.

In general, most of peptide and protein drugs are included in Class 3 in biopharmaceutical classification system (BCS) showing high solubility and low permeability and this results in low absorption rate in the gastrointestinal tract. The peptide and protein drugs show low oral bioavailability because of not only the properties of high hydrophilicity and large molecular weights, but also various gastrointestinal barriers to drug absorption, such as instability at the low pH of gastric acid and enzymatic degradation. In general, the oral bioavailability of the peptide and protein drugs is approximately 0.1%, thereby being unsuitable to be used as a pharmaceutical composition for oral delivery. In order to overcome these limitations, enteric coated dosage forms have been used for bypassing the stomach, but there is a limitation to fundamentally improving the oral absorption of peptide and protein drugs.

On the contrary, biotin moiety conjugation to polypeptide according to an embodiment of the present invention can improve the oral bioavailability. More specifically, covalent coupling with biotin (vitamin B7), which is a type of water-soluble vitamins, can facilitate the transporter-mediated intestinal absorption of polypeptide via the sodium-dependent multivitamin transport (SMVT) system.

Hereinafter, an example according to an embodiment of the present invention will be described in detail.

A biotin moiety-conjugated polypeptide according to an embodiment of the present invention is

a polypeptide with insertions or substitutions of one or more amino acid residue between 9^{th} and 39^{th} in SEQ ID NO:1 with cysteine residue, and

a biotin moiety represented by the following General Formula A is conjugated to cysteine residue of the above polypeptide.

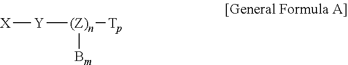

wherein,

X is a functional group capable of conjugation to the polypeptide,

Y is a spacer,

Z is a binding unit,

B may be represented by the following Chemical Formula A-1,

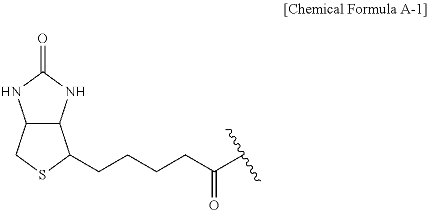 [SMILES: *C(=O)CCCCC1SCC2NC(=O)NC21]

T is a terminal group,

m is an integer of 1 to 10,

n is an integer of 0 or 1 to 10, and

p is an integer of 0 or 1.

The biotin moiety-conjugated polypeptide according to the present invention may be a peptide in which at least one of amino acids 9 to 39 of a polypeptide consisting of an amino acid sequence represented by SEQ ID NO: 1 shown in Table 1 below is substituted or inserted with cysteine. Here, the insertion means that cysteine is inserted before or after at least one amino acid position of the amino acids 9 to 39.

More specifically, the biotin moiety-conjugated polypeptide according to the present invention may be a polypeptide in which at least one of amino acids 9 to 39 of the polypeptide consisting of the amino acid sequence represented by SEQ ID NO: 1 shown in Table 1 below is substituted with cysteine, or cysteine is inserted to the amino acid 39. More specifically, the biotin moiety-conjugated polypeptide may be a polypeptide in which at least one of amino acids 9 to 39 is substituted with cysteine, or cysteine is inserted to the amino acid 39. Much more specifically, the biotin moiety-conjugated polypeptide may be a polypeptide in which cysteine is inserted to the amino acid 39, for example, a polypeptide consisting of an amino acid sequence represented by SEQ ID NO: 2 shown in Table 1 below.

Here, the polypeptide consisting of the amino acid sequence represented by SEQ ID NO: 2 is a polypeptide in which cysteine is inserted after the amino acid position 39 of the polypeptide consisting of the amino acid sequence represented by SEQ ID NO: 1, that is, a polypeptide in which cysteine is inserted to a C-terminal, i.e., an amino acid position 40 of the polypeptide consisting of the amino acid sequence represented by SEQ ID NO: 1.

TABLE 1

| SEQ ID NO: | Peptide sequence |
|---|---|
| 1 | HGEGTFTSDLSKQMEEEAVRLFIEWLKNGGPSSGAPPPS |
| 2 | HGEGTFTSDLSKQMEEEAVRLFIEWLKNGGPSSGAPPPSC |

In general, modifications of one or more amino acids in a specific peptide do not affect a function of the peptide, or, in specific cases, further enhance an ideal function of the original peptide. Actually, it is known that a modified peptide (i.e., a peptide consisting of an amino acid sequence modified (e.g., substituted and/or inserted) with one or more amino acid residues compared to an original reference sequence) retains the biological activity of the original peptide (Mark et al., Proc Natl Acad Sci USA 1984, 81: 5662-6; Zoller and Smith, Nucleic Acids Res 1982, 10: 6487-500). More specifically, it is well known that amino acid sites 1 to 8 as an N-terminal region of exendin-4 are required for GLP-1 receptor binding and biological activity (Eun Ji Park et al., EXPERTOPINION ON THERAPEUTIC PATENTS, 2016, VOL.26, NO.7, 833-842), and it is known that a modification at an inactive site of exendin-4 does not significantly affect the biological activity of the original exendin-4 (Dan Donnelly, British Journal of Pharmacology (2012), 166, 27-41). Therefore, the biotin moiety-conjugated polypeptide according to the present invention may be a polypeptide which includes an amino acid sequence in which at least one of amino acids, for example, amino acids 9 to 39 of the inactive site of exendin-4 consisting of the amino acid sequence of SEQ ID NO: 1 is substituted or inserted with cysteine and has the same biological activity as exendin-4. Moreover, the biotin moiety-conjugated polypeptide according to the present invention may not reduce the activity of exendin-4 by substituting or inserting the amino acid of the inactive site of exendin-4 with cysteine, and accordingly, the polypeptide may have a uniform biological activity.

In addition, the biotin moiety-conjugated polypeptide according to the present invention may have sequence homology of 90%, 91%, 92%, 93%, 94%, 95%, 96%, 97%, 98%, and 99% or more with exendin-4 consisting of the amino acid sequence represented by SEQ ID NO: 1, but is not limited thereto.

In addition, the polypeptide having the sequence homology of 97% or more with exendin-4 consisting of the amino acid sequence represented by SEQ ID NO: 1 may be a polypeptide consisting of an amino acid sequence represented by SEQ ID NO: 2.

In the present specification, “homology” is intended to indicate the degree of similarity with a wild-type amino acid sequence and a wild-type nucleic acid sequence, and comparison of such homology is performed with the naked eye or using a comparison program that is easily purchased. A commercially available computer program may calculate the homology between two or more sequences as a percentage (%). The homology (%) may be calculated on neighboring sequences.

According to an embodiment of the present invention, the biotin moiety may be conjugated by adjusting a conjugating position with the polypeptide. Specifically, in the biotin moiety-conjugated polypeptide according to an embodiment of the present invention, cysteine may be substituted or inserted to at least one of amino acids, for example, amino acids 9 to 39 of the inactive site of exendin-4 in order to adjust the conjugating position with the biotin moiety, and thus, the biotin moiety may be conjugated to the cysteine.

In General Formula A representing the biotin moiety, X is a functional group capable of conjugation to cysteine of the polypeptide. Although not limited thereto, for example, the functional group may be maleimide, amine, succinimide, N-hydroxysuccinimide, aldehyde or carboxyl group, and more specifically maleimide.

In an embodiment of the present invention, when the functional group X is conjugated with cysteine of the polypeptide, the structure may be maintained, or removed or modified.

The Y may be a spacer and may have a structure having cleavability in the body. Although not limited thereto, for example, the Y is a direct-bonded, or substituted or unsubstituted alkylene, wherein the alkylene may include at least one of —O—, —C(═O)NR—, —C(═O)O— or —C(═O)—, —NR—, and —NOR—, and the R may be hydrogen, and substituted or unsubstituted alkyl or aryl.

In one embodiment, the spacer may include a structure represented by the following Formula.

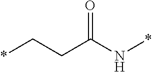 [SMILES: *CCC(=O)N*]

The Z is a binding unit capable of binding to B, and may include, for example, an amino acid, a polypeptide, an alkylene amine, or a polyamidoamine structure, but not limited thereto.

Although not limited thereto, for example, the amino acid may be lysine, 5-hydroxylysine, 4-oxallysine, 4-thialysine, 4-selenalysine, 4-thiahomolysine, 5,5-dimethyllysine, 5,5-difluorolysine, trans-4-dehydrolysine, 2,6-diamino-4-hexynoic acid, cis-4-dehydrolysine, 6-N-methyllysine, diaminopimelic acid, ornithine, 3-methylornithine, a-methylornithine, citrulline or homocitrulline, arginine, aspartate, asparagine, glutamate, glutamine, histidine, ornithine, proline, serine, or threonine.

When then is 0, B may directly bind to Y (spacer).

The T is a terminal group, and although not limited thereto, may be, for example, hydrogen or NH₂.

When the p is 0, the B may be a terminal.

According to an embodiment of the present invention, in General Formula A above, m may be an integer of 1 to 10, and specifically, may be an integer of 1 to 8, 1 to 5, and 1 to 4.

According to an embodiment of the present invention, the biotin moiety may be represented by the following General Formula 1A.

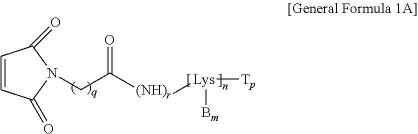

wherein,

Lys is lysine,

T is hydrogen or NH₂,

q is an integer of 1 to 5,

r is an integer of 0, 1 to 3, and

B, n, m, and p are as defined in General Formula A above.

According to an embodiment of the present invention, the biotin moiety may be represented by the following General Formula 2A or 3A.

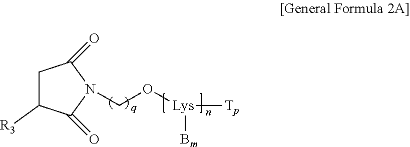

wherein,

Lys is lysine,

T is hydrogen or NH₂,

R₃ is hydrogen or —SO₃—,

q is an integer of 0, or 1 to 4, and

B, n, m, and p are as defined in General Formula A above.

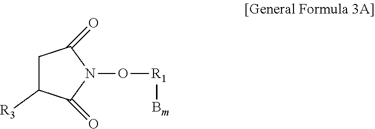

wherein,

R₁ is none or NH,

R₃ is hydrogen or —SO₃—, and

B and m are as defined in General Formula A above.

According to an embodiment of the present invention, the biotin moiety may be represented by the following structure.

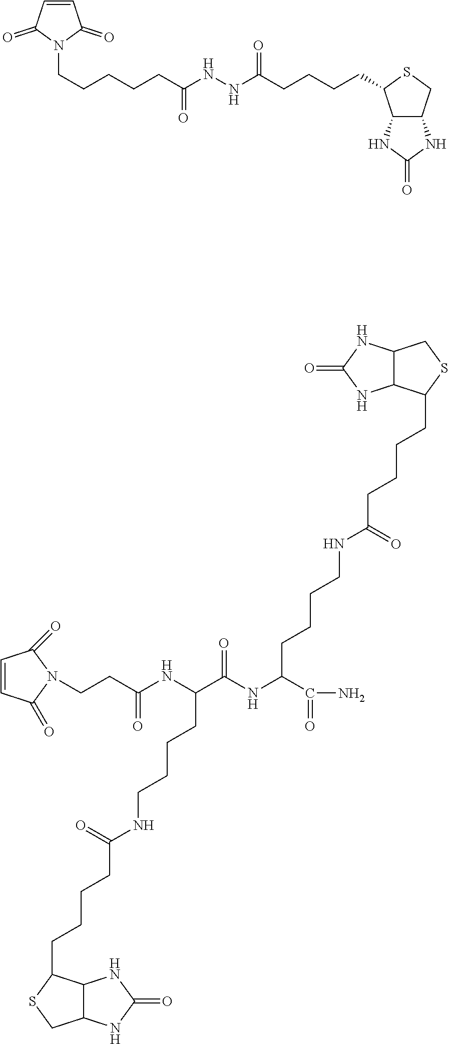 [SMILES: O=C(CCCCCN1C(=O)C=CC1=O)NNC(=O)CCCC[C@@H]1SC[C@@H]2NC(=O)N[C@@H]21 ; NC(=O)C(CCCCNC(=O)CCCCC1SCC2NC(=O)NC21)NC(=O)C(CCCCNC(=O)CCCCC1SCC2NC(=O)NC21)NC(=O)CCN1C(=O)C=CC1=O]

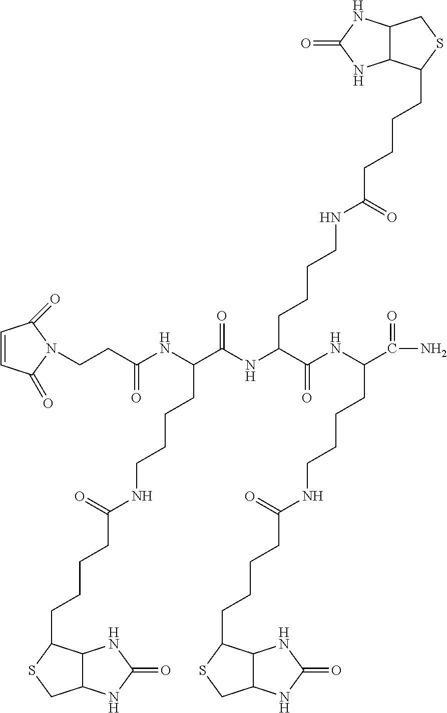 [SMILES: NC(=O)C(CCCCNC(=O)CCCCC1SCC2NC(=O)NC21)NC(=O)C(CCCCNC(=O)CCCCC1SCC2NC(=O)NC21)NC(=O)C(CCCCNC(=O)CCCCC1SCC2NC(=O)NC21)NC(=O)CCN1C(=O)C=CC1=O]

According to an embodiment of the present invention, the polypeptide may be SEQ ID NO:2, and the biotin moiety may be represented by the following structure.

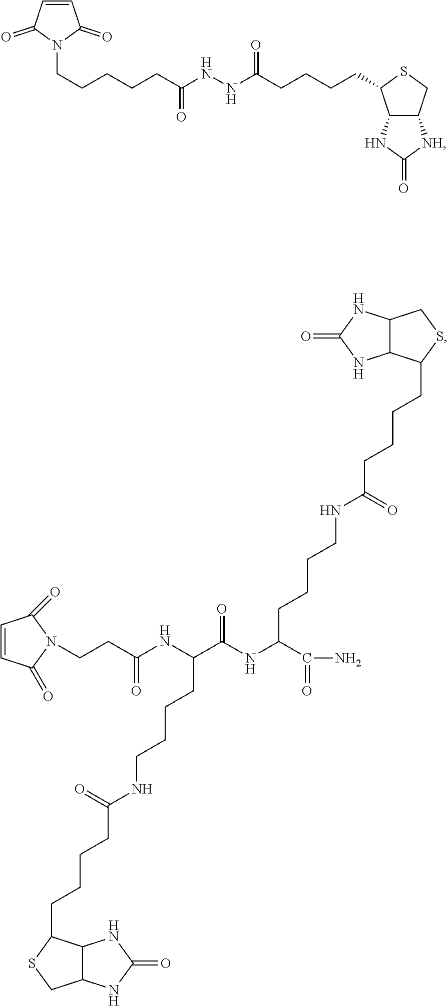 [SMILES: O=C(CCCCCN1C(=O)C=CC1=O)NNC(=O)CCCC[C@@H]1SC[C@@H]2NC(=O)N[C@@H]21 ; NC(=O)C(CCCCNC(=O)CCCCC1SCC2NC(=O)NC21)NC(=O)C(CCCCNC(=O)CCCCC1SCC2NC(=O)NC21)NC(=O)CCN1C(=O)C=CC1=O]

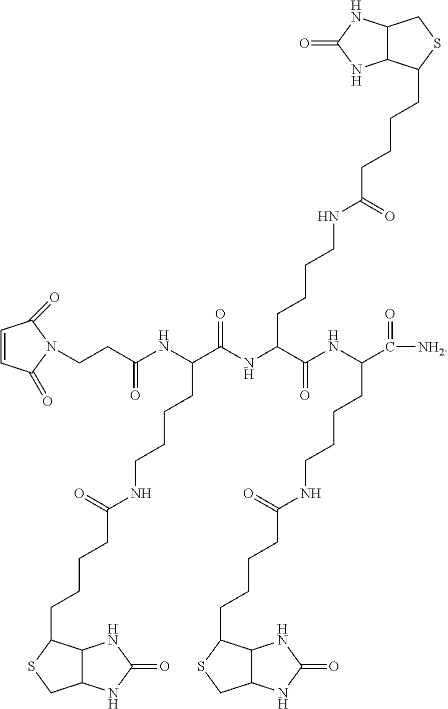 [SMILES: NC(=O)C(CCCCNC(=O)CCCCC1SCC2NC(=O)NC21)NC(=O)C(CCCCNC(=O)CCCCC1SCC2NC(=O)NC21)NC(=O)C(CCCCNC(=O)CCCCC1SCC2NC(=O)NC21)NC(=O)CCN1C(=O)C=CC1=O]

In one embodiment, the conjugating between a biotin moiety and a polypeptide may be represented by Reaction Formula 1 below. In Reaction Formula 1 below,

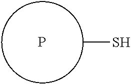

represents a polypeptide, and represents a reaction between General Formula 1A and a thiol group (—SH) which is a cysteine residue present in the polypeptide.

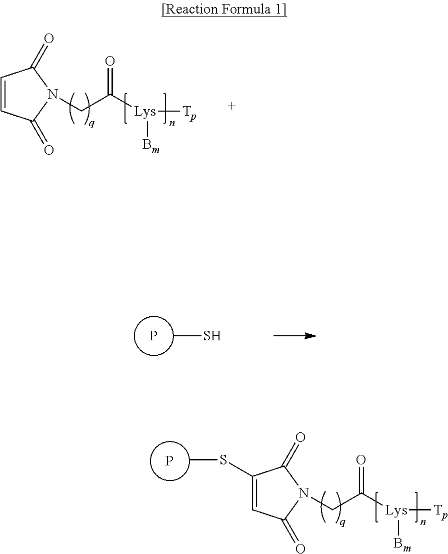

The biotin moiety-conjugated polypeptide according to an embodiment of the present invention may be used as a composition for oral administration, and specifically, may be used as a composition for oral administration for preventing or treating disease caused by insufficient insulin secretion or defective insulin action, obesity, fatty liver disease, or neurodegenerative disease.

Another aspect of the present invention provides a method for preparing a biotin moiety-conjugated polypeptide comprising:

1) obtaining a polypeptide in which at least one of amino acids 9 to 39 of a polypeptide consisting of an amino acid sequence represented by SEQ ID NO: 1 is substituted or inserted with cysteine;

2) obtaining a polypeptide in which a biotin moiety is conjugated to cysteine by reacting the polypeptide obtained in step 1) and the biotin moiety represented by the following General Formula A in an organic solvent; and

3) isolating and purifying the biotin moiety-conjugated polypeptide of step 2);

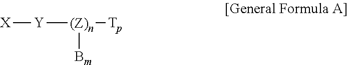

wherein,

X is a functional group capable of conjugation to the polypeptide,

Y is a spacer,

Z is a binding unit,

B may be represented by the following Chemical Formula A-1,

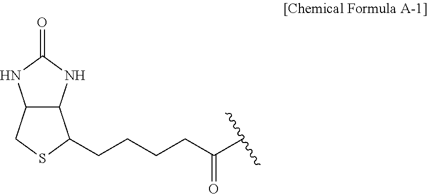 [SMILES: *C(=O)CCCCC1SCC2NC(=O)NC21]

T is a terminal group,

m is an integer of 1 to 10,

n is an integer of 0 or 1 to 10, and

p is an integer of 0 or 1.

The specific description of the polypeptide and the biotin moiety is the same as the description of the biotin moiety-conjugated polypeptide, and the detailed description uses the above contents, and hereinafter, only a configuration specific to the preparation method will be described.

According to an embodiment of the present invention, a well-known technique may be used to obtain the polypeptide in step 1). For example, recombinant DNA technology or chemical synthesis may be used to prepare the polypeptide. The polypeptide may also be isolated from a chemically synthetic reaction product or biologically synthesized from host cells using the recombinant technology. That is, the polypeptide may be purified or isolated so as not to contain other proteins or fragments thereof of host cells or other chemical materials. The polypeptide may be obtained through conventional chemical synthesis that may be applied for synthesis based on a selected amino acid sequence (e.g., [1] Peptide Synthesis, Interscience, New York, 1966, [2] The Proteins, Vol. 2, Academic Press, New York, 1976). On the other hand, the polypeptide may be obtained using any well-known genetic engineering method for preparing the polypeptide (e.g., Morrison J, J Bacteriology 1977, 132: 349-51; Clark-Cu). For example, first, an appropriate vector is prepared by including a polynucleotide encoding the polypeptide in an expressible type (e.g., downstream a regulatory sequence corresponding to a promoter sequence), and transformed into an appropriate host cell. Thereafter, the host cells are incubated so that the polypeptide is produced. In addition, the polypeptide may also be prepared in vitro using an in vitro translation system.

According to an embodiment of the present invention, in step 2), a reaction molar ratio of the biotin moiety to the polypeptide may be 0.5 or more. Specifically, the reaction molar ratio of the biotin moiety to the polypeptide may be 0.5 to 5. The appropriate reaction molar ratio may be selected in consideration of a molecular structure of the biotin moiety, a molecular weight, solubility, a pH of a reaction solution, a reaction temperature, a reaction time, and the like.

In addition, the reaction in step 2) may be performed using a buffer solution or an organic solvent. The buffer solution or the organic solvent is not particularly limited, and a buffer solution commonly used in the art may be appropriately selected according to the structure of the biotin moiety.

In addition, the temperature and time of the reaction in step 2) may be appropriately adjusted according to the characteristics of the biotin moiety and the polypeptide to be used. Although not limited thereto, for example, the reaction in step 2) may be performed at 4° C. for 3 hours or more, and may be performed at room temperature for a shorter time. Specifically, the reaction in step 2) may be performed at room temperature for 10 to 160 minutes, more specifically at room temperature for 20 to 140 minutes, and much more specifically at room temperature for 30 to 120 minutes. The temperature and time of the reaction may be related to the degree of reactivity of the biotin moiety to be used. When an appropriate reaction time has elapsed, the reaction may be stopped by lowering the pH of the reaction solution.

In addition, a step of removing an unreacted material after the reaction in step 2) may be performed. The method of removing the unreacted material may be performed by a method commonly used in the art. Although not limited thereto, for example, the unreacted material may be removed by dialysis or the like using a suitable buffer solution, for example, a solution such as phosphate buffered saline (PBS).

According to an embodiment of the present invention, the isolation and purification in step 3) may be performed using size exclusion chromatography, reverse phase high performance liquid chromatography, ion exchange chromatography, or the like, but is not limited thereto.

Another aspect of the present invention provides a pharmaceutical composition for oral administration comprising the biotin moiety-conjugated polypeptide described above. The biotin moiety-conjugated polypeptide according to an embodiment of the present invention is conjugated with biotin (vitamin B7), which is a type of water-soluble vitamin, to be absorbed by active transport through a sodium-dependent multivitamin transporter and penetrates into the intestinal membrane to improve the bioavailability in the gastrointestinal tract.

Yet another aspect of the present invention provides a pharmaceutical composition comprising the biotin moiety-conjugated polypeptide described above. The pharmaceutical composition is a pharmaceutical composition that can be administered orally.

In the biotin moiety-conjugated polypeptide according to an embodiment of the present invention, since cysteine is substituted or inserted to the inactive site of exendin-4 for conjugating the biotin moiety, the activity of exendin-4 may not be inhibited.

According to an embodiment of the present invention, it is possible to provide a pharmaceutical composition for preventing or treating diabetes, comprising the biotin moiety-conjugated polypeptide described above. The pharmaceutical composition is a pharmaceutical composition for oral administration.

The diabetes may include Type I diabetes, Type II diabetes and/or diabetic complications.

According to an embodiment of the present invention, it is possible to provide a pharmaceutical composition for preventing or treating obesity, comprising the biotin moiety-conjugated polypeptide described above. The pharmaceutical composition is a pharmaceutical composition for oral administration.

According to an embodiment of the present invention, it is possible to provide a pharmaceutical composition for preventing or treating fatty liver disease, comprising the biotin moiety-conjugated polypeptide described above. The pharmaceutical composition is a pharmaceutical composition for oral administration.

More specifically, the fatty liver disease may be simple fatty liver disease, non-alcoholic fatty liver disease, nutritional fatty liver disease, starvation fatty liver disease, obesity fatty liver disease, diabetic fatty liver disease, steatohepatitis, liver fibrosis, liver sclerosis and/or cirrhosis.

According to an embodiment of the present invention, it is possible to provide a pharmaceutical composition for preventing or treating irritable bowel syndrome, comprising the biotin moiety-conjugated polypeptide described above. The pharmaceutical composition is a pharmaceutical composition for oral administration.

The irritable bowel syndrome may be caused by a decrease in plasma glucose, suppression of gastric or intestinal movement, suppression of stomach or intestinal fasting, or suppression of food intake.

According to an embodiment of the present invention, it is possible to provide a pharmaceutical composition for preventing or treating neurodegenerative disease, comprising the biotin moiety-conjugated polypeptide described above. The pharmaceutical composition is a pharmaceutical composition for oral administration.

The neurodegenerative diseases may be, more specifically, Alzheimer's disease, Parkinson's disease, progressive supranuclear palsy (PSP), multiple system atrophy (MSA), Lewy body dementia, Parkinson's disease dementia epilepsy, stroke, Huntington's chorea, cerebral hypoxia, multiple sclerosis and/or peripheral neuropathy.

Another aspect of the present invention provides a method for preventing or treating for diabetes, obesity, fatty liver disease, irritable bowel syndrome or neurodegenerative disease in a subject comprising administering the biotin moiety-conjugated polypeptide described above.

The specific description of the diabetes, fatty liver disease, irritable bowel syndrome or neurodegenerative disease is the same as the description above.

According to an embodiment of the present invention, the pharmaceutical composition may be formulated and administered in various oral or parenteral dosage forms, but is not limited thereto.

When the pharmaceutical composition is formulated, the formulation may be prepared by using a diluent or an excipient, such as a filler, a solubilizing agent, an extender, a binder, a wetting agent, a disintegrating agent, and a surfactant which are generally used.

A solid formulation for oral administration includes a tablet, a pill, a powder, a granule, a capsule, and the like, and the solid formulation may be prepared by mixing at least one excipient, for example, starch, calcium carbonate, sucrose or lactose, gelatin, and the like with the compound.

Further, lubricants such as magnesium stearate talc may be used in addition to simple excipients. A liquid formulation for oral administration may correspond to a suspension, an oral liquid, an emulsion, a syrup, and the like, and may include various excipients, for example, a wetting agent, a sweetener, an aromatic agent, a preserving agent, and the like, in addition to water and liquid paraffin which are commonly used as simple diluents.

A formulation for parenteral administration includes a sterile aqueous solution, a non-aqueous solution, a suspension, an emulsion, a lyophilizing agent, and a suppository. As the non-aqueous solution and the suspension, propylene glycol, PEG, vegetable oil such as olive oil, injectable ester such as ethyl oleate, and the like may be used.

In addition, calcium or vitamin D3 may be added to improve efficacy as a therapeutic agent for proliferative diseases or autoimmune diseases.

The dose range of the pharmaceutical composition according to an embodiment of the present invention may vary depending on the patient's weight, age, sex, health condition, diet, administration time, administration method, excretion rate, and disease severity. However, generally, the pharmaceutical composition may be administered once or several times a day within a range of effective daily dose. In addition, it is possible to administer an effective dose several times every 1 to 2 weeks.

Hereinafter, the present invention will be described in detail by Examples and Experimental Examples. However, the following Examples and Experimental Examples are just illustrative of the present invention, and the contents of the present invention are not limited to the following Examples and Experimental Examples.

### EXAMPLE

### Examples 1 to 3: Preparation of Biotin Moiety-Conjugated Polypeptide

A polypeptide conjugated with a biotin moiety to cysteine residue was prepared as follows.

Exendin-4 represented by SEQ ID NO: 1 (HGEGTFTSDLSKQMEEEAVRLFIEWLKNGGPSSGAPPPS) does not contain cysteine. Exendin-4 is well-known that the N-terminal domain play an important role in receptor binding and the cellular activity. Therefore, a polypeptide has amino acid sequence which one or more amino acid residue between 9^{th} and 39^{th} in Exendin-4 sequence was inserted or substituted by cysteine residue to conjugate with biotin moiety. A polypeptide of SEQ ID NO: 2 in which cystine was inserted after 39^{th} serine residue of Exendin-4 to conjugate the biotin moiety was obtained from CS Bio Co., Ltd.

Different types of Biotin moiety shown in Table 2 and a polypeptide of SEQ ID NO: 2 was reacted with gently mixing at room temperature for 30 minutes or more. Molar ratio for a polypeptide and biotin moiety was 1:2 and reaction solvent was 0.3% triethylamine in dimethyl sulfoxide. The reaction was then stopped by adding a 1% trifluoroacetic acid.

TABLE 2

| | Polypeptide | Biotin moiety | |
|---|---|---|---|
| Example 1 | HGEGTFTSDLSK | B1 | N-Biotinoyl-N′- |
| | QMEEEAVRLFIE | | (6-maleimidohexanoyl)hydrazide |
| Example 2 | WLKNGGPSSGA | B2 | 3-Maleimidopropionate-Lys(Biotin)- |
| | PPPSC (SEQ | | Lys(Biotin)-CONH₂ |
| Example 3 | ID NO: 2) | B3 | 3-Maleimidopropionate-Lys(Biotin)- |
| | | | Lys(Biotin)-Lys(Biotin)-CONH₂ |

### Comparative Example 1: Preparation of Biotin Moiety-Conjugated Exendin-4

Biotinylated exendin-4, which is a polypeptide with biotin moiety conjugated to N-terminus, lysine¹² and lysine²⁷ of exendin-4, was prepared as follows; 1 mL of biotin-N-hydroxysuccinimide ester (Sigma, Saint Louis, Mo.) was mixed with the same volume of exendin-4 of SEQ ID NO: 1 in dimethyl sulfoxide containing 0.3% triethylamine. The mixture with molar ratio 1:2 to 1:3 (exendin-4:biotin moiety) was gently shaking at room temperature for 60 minutes and the reaction was stopped by adding a 1% trifluoroacetic acid.

Biotinylated exendin-4 represented by comparative example 1 was isolated and purified from reaction mixtures by reversed phase-high performance liquid chromatography (RP-HPLC). Capcell-pak RP-18 (250×4 mm, 5 mm, Shiseido, Japan) was used as a column for isolation, and mobile phase consisted of 0.1% TFA in deionized water (eluent A) and acetonitrile containing 0.1% TFA (eluent B). Linear gradient from 36% to 41% of eluent B was administered over 20 minutes with a flow rate of 1 mL/min. HPLC fractions corresponding to peaks were monitored at 280 nm and collected and flushed with nitrogen. Biotinylated exendin-4 was obtained and characterized by HPLC and MALDI-TOF mass spectrometry.

### Experimental Example 1: Isolation/Purification and Confirmation of Biotin Moiety-Conjugated Polypeptide

polypeptides conjugated with biotin moiety represented in Examples 1 to 3 were isolated and purified by preparative RP-HPLC using a SUPERSIL ODS-1 column (10×250 mm, 5 mm, LB Science, South Korea)at ambient temperature.

Mobile phase was consisted of 0.1% TFA in deionized water (eluent A) and acetonitrile containing 0.1% TFA (eluent B). Linear gradient from 30% to 50% of eluent B was administered over 20 minutes with a flow rate of 4.7 mL/min. HPLC fractions corresponding to peaks were monitored at 280 nm and collected.

The collected peaks were concentrated and purified using an ultra-centrifugal filter having an appropriate molecular weight cut-off after removing an organic solvent and TFA under vacuum. Evaporation of an organic solvent and TFA under vacuum. The purity of prepared polypeptide was confirmed by RP-HPLC analysis. Analysis was performed using a Gemini C18 column (4.6×250 mm, 5 mm; Phenomenex, Calif., USA) at 25° C. by a gradient elution using a mobile phase of 0.1% TFA in deionized water (eluent A) and acetonitrile containing 0.1% TFA (eluent B) at a flow rate of 1 mL/min. The elution was used by linear gradient from 30% to 50% of eluent B over 20 minutes. The UV absorbance was monitored at 280 nm.

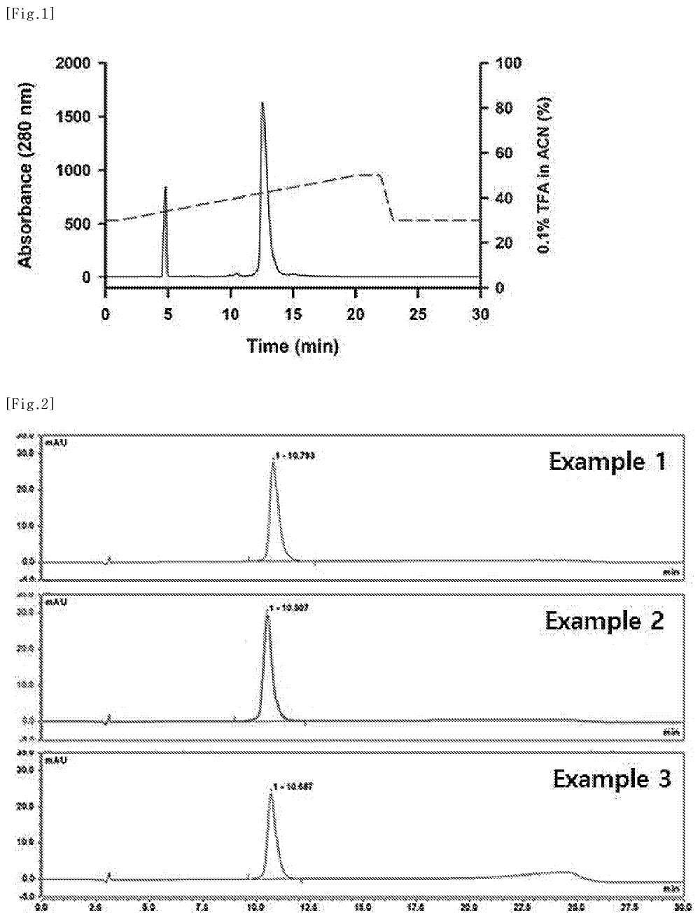

FIG. 1 is a purification chromatogram of the biotin moiety-conjugated polypeptide of Example 3.

FIG. 2 is HPLC chromatograms of the biotin moiety-conjugated polypeptides in Examples 1 to 3.

As a result of RP-HPLC analysis, the purity of biotin moiety-conjugated polypeptide represented in Examples 1 to 3 was determined more than 99%.

### Experimental Example 2: Characterization of Biotin Moiety-Conjugated Polypeptides

MALDI-TOF mass spectrometry for measuring the molecular masses of biotin moiety-conjugated polypeptides was carried out in a Bruker Daltonics Microflex MALDI-TOF mass spectrometer (Bremen, Germany) with 337 nm nitrogen laser. A saturated solution of a-Cyano-4-hydroxycinnamic acid in 50% acetonitrile containing 0.1% TFA was used as a matrix solution. Each analyte was mixed with the matrix solution at a ratio of analyte:matrix=1:1 (v/v) and then 1μL of the analyte-matrix solution was deposited onto the sample plate and dried by vacuum evaporation. Mass spectra were obtained in the linear and positive-ion mode.

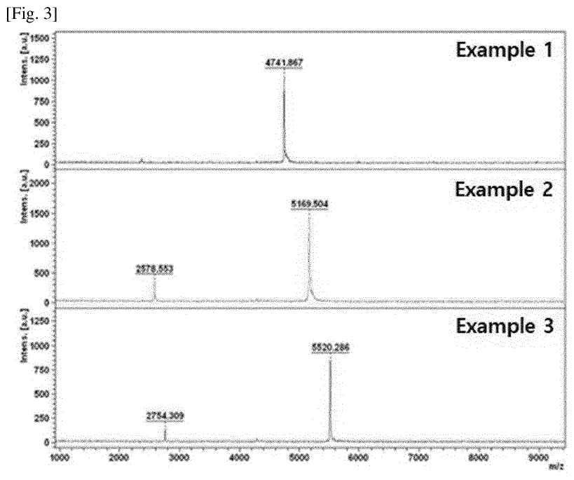

FIG. 3 shows MALDI-TOF mass spectra of the biotin moiety-conjugated polypeptides in Examples 1 to 3. Measured molecular masses were consistent with the molecular masses expected after conjugation with biotin moiety and polypeptide represented SEQ ID NO: 2.

### Experimental Example 3: Identification of Modification Site of Biotin Moiety in Example 1 to 3

In order to confirm the biotin conjugation sites, the biotin moiety-conjugated polypeptides in Examples 1 to 3 and polypeptide of SEQ ID NO: 1 were digested with an endoproteinase Lys-C, which cleaves peptide bonds at the carboxyl side of lysine. A certain amount of Lys-C was added to 1 mg/mL of biotin moiety-conjugated polypeptide in phosphate buffer (10 mM; pH 7.4) and the enzymatic digestion was allowed to continue for 4 hours at 37° C. The Lys-C digests were directly analyzed by RP-HPLC and peaks shown in RP-HPLC were collected and analyzed by MALDI-TOF MS. The mass spectra between SEQ ID NO: 1 and biotin moiety-conjugated polypeptides after Lys-C digestion were compared for identifying the biotin moiety conjugation site. Table 3 shows RP-HPLC analysis condition and analytical condition for MALDI-TOF mass spectrometry is the same to method described in Experimental Example 2.

TABLE 3
HPLC condition

| | |
|---|---|
| Column | Gemini C18 Column (4.6 × 250 mm, 5 mm) |
| Column temperature | 25° C. |
| Mobile phase: | Solvent A: Distilled water added with 0.1% TFA |
| | Solvent B: Acetonitrile added with 0.1% TFA |
| Gradient | 10% to 80% (% B) for 50 min |
| UV Absorbance | 215 & 280 nm |

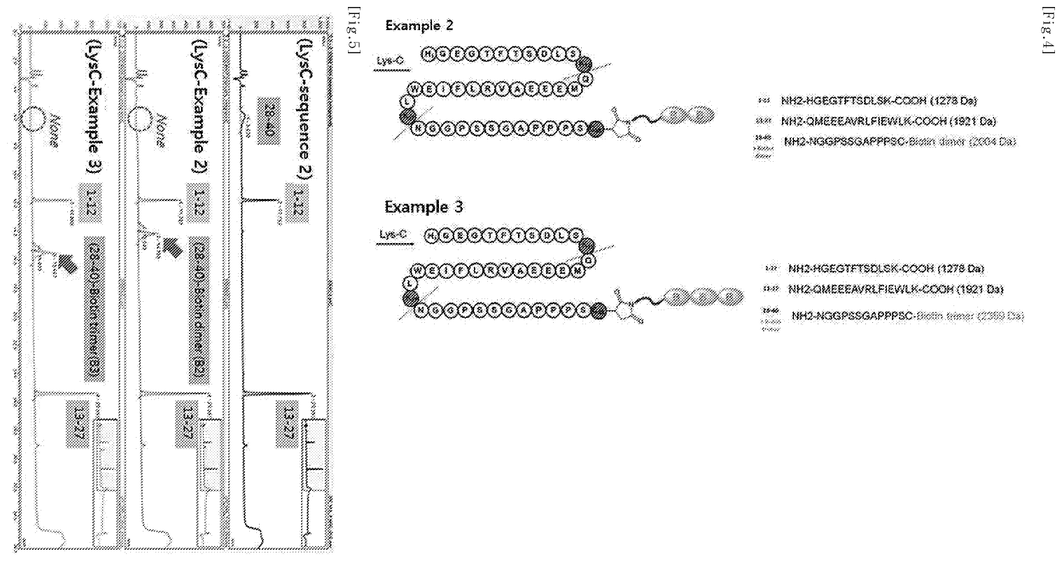

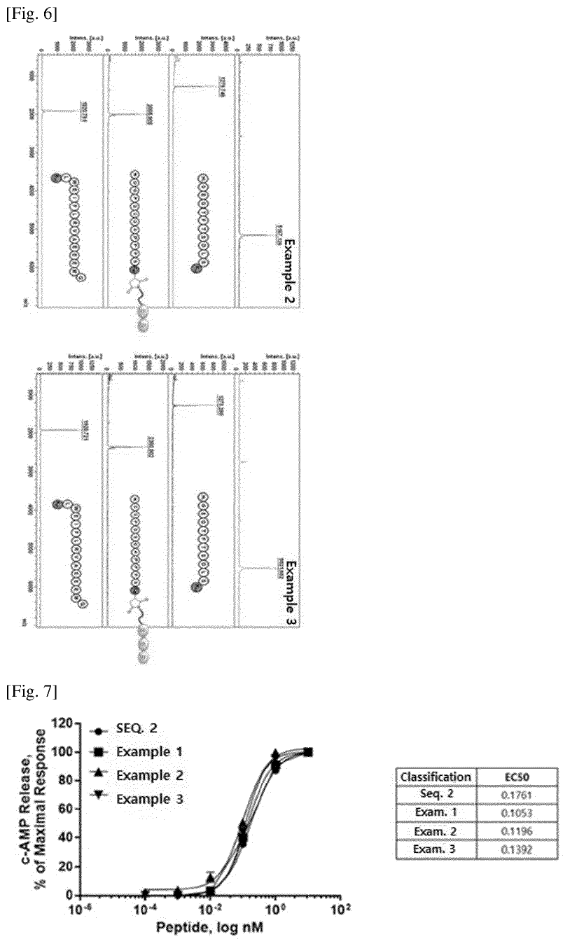

FIG. 4 shows a schematic diagram demonstrating cleavage after Lys-C digestion to identify the biotin moiety conjugated site of polypeptides in Example 2 and 3. FIG. 5 and 6 show RP-HPLC chromatogram and MALDI-TOF MS results for the site identification. HPLC peak for peptide fragment corresponding to sequence (28-40)was not observed in HPLC chromatogram of Lys-C digested polypeptides for Example 2 and 3. Additional peak for biotin moiety-conjugated sequence (28-40)was observed and their molecular masses were identified by MALDI-TOF mass spectrometry (Table 4). This result indicates that the conjugation site of biotin moiety was the cysteine⁴° residue in polypeptides of Example 2 and 3.

TABLE 4

| | Observed mass (m/z) | | | |
|---|---|---|---|---|
| | by MALDI-TOF MS | | | |
| | | Lys-C | Retention | Assigned sequence |
| | Intact | digested | time in | of Lys-C digested |
| Example | samples | samples | HPLC | fragments |
| Example 2 | 5167.1 | 1279.7 | 11.8 min | His1-Lys12 |
| | | 2005.9 | 14.2 min | Asn28-Cys40-B2 |
| | | 1920.8 | 25.4 min | Gln13-Lys27 |
| Example 3 | 5521.7 | 1278.3 | 11.8 min | His1-Lys12 |
| | | 2360.6 | 15.5 min | Asn28-Cys40-B3 |
| | | 1920.7 | 25.4 min | Gln13-Lys27 |

### Experimental Example 4: Reaction and Production Yield of Biotin Moiety-Conjugated Polypeptide

The reaction and production yields of the biotin moiety-conjugated polypeptides of

Examples 1 to 3 were compared with biotinylated exendin-4 in Comparative Example 1.

The reaction yield was determined by peak area changes at 280 nm in RP-HPLC before and after biotin conjugation reaction.

The production yield was determined by dividing the mole of biotin moiety-conjugated polypeptide by mole of intact polypeptide added to the reaction. These results are summarized in Table 5.

TABLE 5

| | Comparative Example 1 | Examples 1 to 3 |
|---|---|---|
| Biotin moiety con- | N-terminal and | C-terminal |
| jugating site | lysine of Exendin-4 | cysteine |
| Reaction yield | 5% to 35% | >99% |
| Production yield | 5% to 30% | 80% |

As shown in Table 5, reaction and production yields of Comparative Example 1 were low and inconsistent because biotin moiety could react with amine of N-terminal and lysine and produce the various isomers. In contrast, conjugation of cysteine residue by biotin moiety resulted in a dramatic increase in the reaction and production yields shown in Example 1 to 3.

### Experimental Example 5: Measurement of Biological Activity of Biotin Moiety-Conjugated Polypeptide

The biological activities of the polypeptide before and after conjugation of the biotin moiety were compared. In addition, biological activities according to the conjugating site of the biotin moiety were compared.

Specifically, HEK293/CRE-Luc/GLP1R (Genescript, #M00562) cells were dispensed into a 96-well plate at 50,000 cells per well, and then incubated in a DMEM medium (containing 10% FBS, 400 mg/mL of G418, and 200 mg/mL of Hygromycin B) for 24 hours. Then, the culture media was removed, and each drug was added at a concentration of 100, 10, 1, 0.1, 0.01, 0.001, and 0.0001 nM by 100 ml per well. After the incubation time of 2 hours, 100 ml of a luciferase reagent was added and reacted for 3 minutes, and then luminescence was measured with a 96-well microplate reader.

FIG. 7 is a graph showing the biological activities of the polypeptides of Examples 1 to 3 and a polypeptide (polypeptide consisting of an amino acid sequence represented by SEQ ID NO: 2) before conjugation with a biotin moiety. As illustrated in FIG. 7, it was confirmed that the biological activities of the polypeptides of Examples 1 to 3 in which the biotin moiety was conjugated to cysteine of the polypeptide were equal to that of a polypeptide (Sequence 2) consisting of an amino acid sequence represented by SEQ ID NO: 2 in which cysteine was inserted to a C-terminal as an inactive site (amino acid sequence sites 9 to 39) of exendin-4. Through this, it can be seen that the conjugation of the biotin moiety does not affect the biological activity of the peptide.

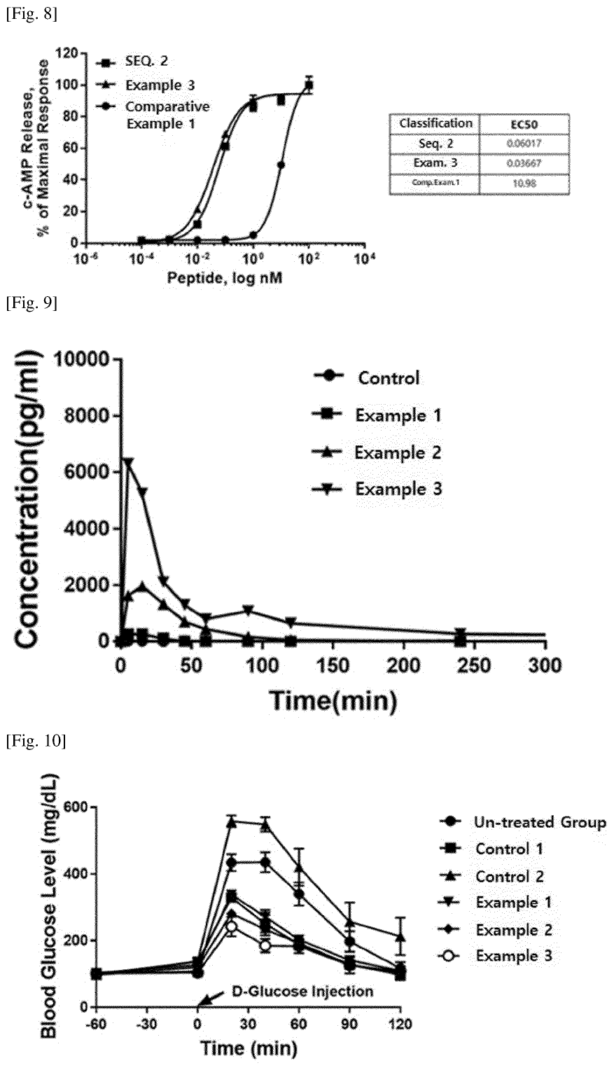

FIG. 8 is a graph showing biological activities of the polypeptide of Comparative Example 1, the polypeptide of Example 3, and a polypeptide before conjugation with a biotin moiety thereof. As illustrated in FIG. 8, it was confirmed that compared to the polypeptide of Comparative Example 1 in which the biotin moiety was conjugated to the N-terminal and lysine residues, the polypeptide of Example 3 conjugated to the cysteine residue of the C-terminal as an inactive site had excellent biological activity. Through this, it can be seen that when the biotin moiety is conjugated to the active site of exendin-4 and the lysine residue, the biological activity is reduced.

### Experimental Example 6: Measurement of Oral Bioavailability of Biotin Moiety-Conjugated Polypeptide

The pharmacokinetic profiles were compared with each other to confirm the oral bioavailability.

After the biotin moiety-conjugated polypeptides prepared in Examples 1 to 3 were orally administered in an amount of 500 mg/kg to experimental rats (SD rat) having a body weight of about 200 g, the blood was collected from jugular vein and drug concentration changes in blood over time were measured by an enzyme-linked immunosorbent assay. In the case of a control, exendin-4 consisting of an amino acid sequence represented by SEQ ID NO: 1 was administered orally in an amount of 100 mg/kg, and then drug concentration changes in blood over time were measured in the same manner as described above. The results were calculated as an average value and shown in Table 6 below.

On the other hand, exendin-4 has 97% homology with the polypeptide represented by SEQ ID NO: 2, and is hardly absorbed orally, and thus, exendin-4 is well known as a GLP-1 agonist that is required to be administered by intravenous or subcutaneous injection.

TABLE 6

| | Control | Example 1 | Example 2 | Example 3 |
|---|---|---|---|---|
| Oral bioavailability | 0.03 | 7.7 | 8.1 | 16.7 |
| (%) | | | | |

FIG. 9 is a graph showing blood concentrations over time after oral administration to rats of Examples 1 to 3. As illustrated in Table 6 and FIG. 9, it was confirmed that the biotin moiety-conjugated polypeptides of Examples 1 to 3 exhibited an excellent oral bioavailability compared to the control.

### Experimental Example 7: Measurement of Blood Glucose Regulating Ability of Biotin Moiety-Conjugated Polypeptide

In order to confirm the blood glucose control efficacy, an intraperitoneal glucose tolerance test (IPGTT) was conducted after oral administration of Examples 1 to 3 of an oral GLP-1 agonist formulation to mice.

In order to measure the intraperitoneal glucose tolerance in an animal model, 100 ml (10 ug/mouse) of the biotin moiety-conjugated polypeptides prepared in Examples 1 to 3 above were orally administered to 9-week-old male mice (C57BL/6) at −60 minutes, then 200 ml of glucose (2 g/kg) was intraperitoneally injected, and glucose change in the blood collected from the tail vein were observed at −60, 0, 20, 40, 60, 90, and 120 minutes. On the other hand, exendin-4 consisting of an amino acid sequence represented by SEQ ID NO: 1 that was subcutaneously administered was used as Control 1, and that was orally administered was used as Control 2.

FIG. 10 is a graph showing changes in blood glucose after administering glucose to each sample. As illustrated in FIG. 10, it was confirmed that a glucose reducing effect was shown in Control 1 of subcutaneous administration as compared to Control 2 and a non-treated group. Through this, it can be seen that exendin-4 to which the biotin moiety is not conjugated is hardly absorbed oral administration and required to be administered by intravenous or subcutaneous injection.

On the other hand, it was confirmed that the biotin moiety-conjugated polypeptides of Examples 1 to 3 exhibited improved glucose control ability due to an excellent oral bioavailability.

As described above, in the biotin moiety-conjugated polypeptide according to an embodiment of the present invention, it can be seen that the biotin moiety is conjugated only to the inserted cysteine to produce a uniform material without forming isomers, thereby improving the reaction rate and yield.

In addition, it can be seen that the biotin moiety-conjugated polypeptide according to the present invention exhibits an excellent oral bioavailability to be used as a pharmaceutical composition for oral administration, and can increase an oral bioavailability of drugs.

In addition, in the biotin moiety-conjugated polypeptide according to the present invention, since cysteine was inserted to the C-terminal as the inactive site of exendin-4 to conjugate the biotin moiety, it is expected to have an effect of preventing or treating obesity, fatty liver disease, and neurodegenerative disease in addition to an effect of preventing or treating diabetes due to 97% homology with exendin-4 without reducing the biological activity of the polypeptide.

According to an embodiment of the present invention, a polypeptide in which a biotin moiety is selectively conjugated to cysteine is prepared according to the method of the present invention using a polypeptide in which cysteine is inserted to a C-terminal, which is an inactive site of exendin-4, and the biotin moiety, thereby producing a uniform material without formation of isomers and improving the reaction rate and yield. In addition, the biotin moiety may be conjugated to cysteine without affecting the biological activity so as not to reduce the biological activity of the polypeptide.

The biotin moiety-conjugated polypeptide according to an embodiment of the present invention may have an excellent oral bioavailability.

The biotin moiety-conjugated polypeptide according to an embodiment of the present invention may prevent the polypeptide from being degraded from enzymes, and ultimately penetrates into an intestinal membrane through in vivo transporters to improve the bioavailability in the gastrointestinal tract.

The biotin moiety-conjugated polypeptide according to an embodiment of the present invention is conjugated with biotin, which is a type of water-soluble vitamin B7, to be absorbed by active transport through a sodium-dependent multivitamin transporter.

Therefore, the biotin moiety-conjugated polypeptide according to an embodiment of the present invention may be used as a composition for oral administration, and specifically, may be used as a composition for oral administration for preventing or treating diabetes, obesity, fatty liver disease, irritable bowel syndrome, or neurodegenerative disease.

Those skilled in the art will recognize or confirm a number of equivalents to the specific embodiments of the invention described herein by using only routine experiments. Such equivalents are intended to be included in the following claims. The aforementioned description of the present invention is to be exemplary, and it can be understood by those skilled in the art that it can be easily modified in other detailed forms without changing the technical spirit or required features of the present invention. Therefore, it should be appreciated that the aforementioned embodiments are illustrative in all aspects and are not restricted. For example, respective components described as single types can be distributed and implemented, and similarly, components described to be distributed can also be implemented in a combined form. Further, it is to be understood that all changes or modifications derived from the meaning and scope of the appended claims and equivalent concepts thereof are included in the scope of the present invention.

While the present invention has been described with respect to the specific embodiments, it will be apparent to those skilled in the art that various changes and modifications may be made without departing from the spirit and scope of the invention as defined in the following claims.

### INDUSTRIAL APPLICABILITY

The biotin moiety-conjugated polypeptide according to an embodiment of the present invention may be used as a composition for oral administration, and specifically, may be used as a composition for oral administration for preventing or treating diabetes, obesity, fatty liver disease, or neurodegenerative disease.

Sequence Listing Free Text
SEQ ID NO: 1:
HGEGTFTSDLSKQMEEEAVRLFIEWLKNGGPSSGAPPPS
SEQ ID NO: 2:
HGEGTFTSDLSKQMEEEAVRLFIEWLKNGGPSSGAPPPSC

1. A biotin moiety-conjugated polypeptide, wherein
the polypeptide is a polypeptide in which at least one of amino acids 9 to 39 of a polypeptide consisting of an amino acid sequence represented by SEQ ID NO: 1 is substituted or inserted with cysteine, and
a biotin moiety represented by the following General Formula A is conjugated to cysteine of the polypeptide; 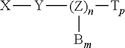
wherein,
X is a functional group capable of being conjugated to the polypeptide,
Y is a spacer,
Z is a binding unit,
B may be represented by the following Chemical Formula A-1, 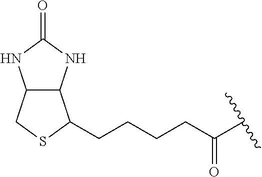 [SMILES: *C(=O)CCCCC1SCC2NC(=O)NC21]
T is a terminal group,
m is an integer of 1 to 10,
n is an integer of 1 to 10, and
p is an integer of 0 or 1.

2. The biotin moiety-conjugated polypeptide of claim 1, wherein the polypeptide is a polypeptide in which at least one of amino acids 9 to 39 of a polypeptide consisting of an amino acid sequence represented by SEQ ID NO: 1 is substituted with cysteine or cysteine is inserted to the amino acid 39.

3. The biotin moiety-conjugated polypeptide of claim 2, wherein the polypeptide in which cysteine is inserted to the amino acid 39 is a polypeptide consisting of an amino acid sequence represented by SEQ ID NO: 2.

4. The biotin moiety-conjugated polypeptide of claim 1, wherein the functional group includes maleimide, amine, succinimide, N-hydroxysuccinimide, aldehyde or carboxyl group.

5. The biotin moiety-conjugated polypeptide of claim 1, wherein the spacer is a direct-bonded, or substituted or unsubstituted alkylene, wherein the alkylene includes at least one of —O—, —C(═O)NR—, —C(═O)O— or —C(═O)—, —NR—, and —NOR—, and the R is hydrogen, and substituted or unsubstituted alkyl or aryl.

6. The biotin moiety-conjugated polypeptide of claim 1, wherein the binding unit includes an amino acid, a polypeptide, an alkylene amine, or a polyamidoamine structure.

7. The biotin moiety-conjugated polypeptide of claim 1, wherein the biotin moiety is represented by the following General Formula 1A; 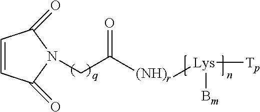
wherein,
Lys is lysine,
T is hydrogen or NH_{2,}
q is an integer of 1 to 5,
r is an integer of 0, 1 to 3, and
B, n, m, and p are as defined in General Formula A above.

8. The biotin moiety-conjugated polypeptide of claim 1, wherein the biotin moiety is represented by the following General Formula 2A; 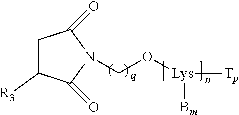
wherein,
Lys is lysine,
T is hydrogen or NH_{2,}
R₃ is hydrogen or —SO₃—,
q is an integer of 0, or 1 to 4, and
B, n, m, and p are as defined in General Formula A above.

9. The biotin moiety-conjugated polypeptide of claim 1, wherein the biotin moiety is represented by the following General Formula 3A; 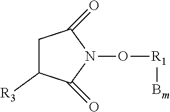
wherein,
R₁ is a direct bond or NH,
R₃ is hydrogen or —SO₃—, and
B and m are as defined in General Formula A above.

10. The biotin moiety-conjugated polypeptide of claim 1, wherein the biotin moiety and the polypeptide are conjugated by a thiol-ether bond or an amide bond.

11. The biotin moiety-conjugated polypeptide of claim 1, wherein the biotin moiety is represented by any one of the following Formulas;

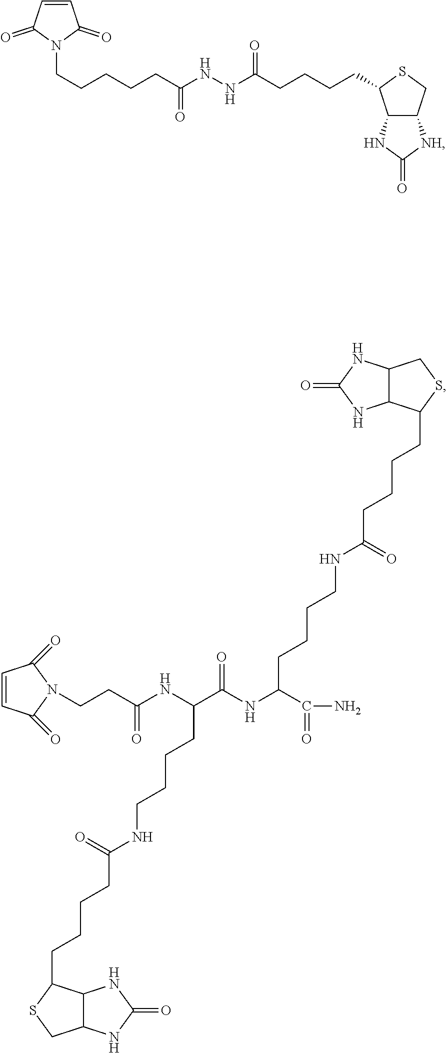 [SMILES: O=C(CCCCCN1C(=O)C=CC1=O)NNC(=O)CCCC[C@@H]1SC[C@@H]2NC(=O)N[C@@H]21 ; NC(=O)C(CCCCNC(=O)CCCCC1SCC2NC(=O)NC21)NC(=O)C(CCCCNC(=O)CCCCC1SCC2NC(=O)NC21)NC(=O)CCN1C(=O)C=CC1=O]

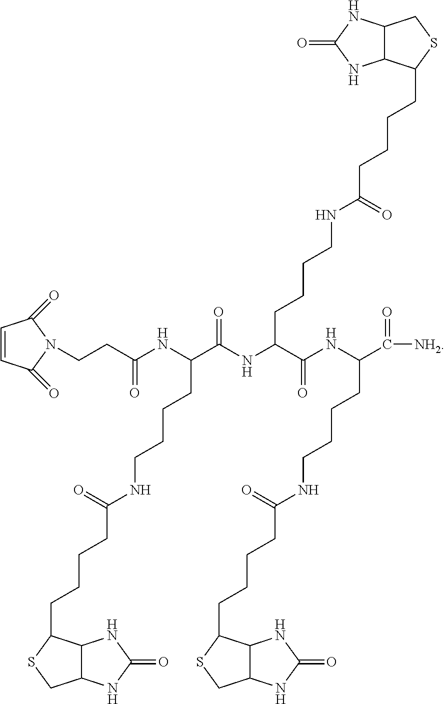 [SMILES: NC(=O)C(CCCCNC(=O)CCCCC1SCC2NC(=O)NC21)NC(=O)C(CCCCNC(=O)CCCCC1SCC2NC(=O)NC21)NC(=O)C(CCCCNC(=O)CCCCC1SCC2NC(=O)NC21)NC(=O)CCN1C(=O)C=CC1=O]

12. A method for preparing a biotin moiety-conjugated polypeptide comprising:
1) obtaining a polypeptide in which at least one of amino acids 9 to 39 of a polypeptide consisting of an amino acid sequence represented by SEQ ID NO: 1 is substituted or inserted with cysteine;
2) obtaining a polypeptide in which a biotin moiety is conjugated to cysteine by adding the polypeptide obtained in step 1) and the biotin moiety represented by the following General Formula A to an organic solvent and reacting; and
3) isolating and purifying the biotin moiety-conjugated polypeptide of step 2). 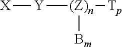
wherein,
X is a functional group capable of being conjugated to the polypeptide,
Y is a spacer,
Z is a binding unit,
B may be represented by the following Chemical Formula A-1, 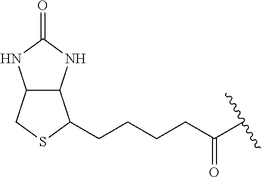 [SMILES: *C(=O)CCCCC1SCC2NC(=O)NC21]
T is a terminal group,
m is an integer of 1 to 10,
n is an integer of 1 to 10, and
p is an integer of 0 or 1.

13. A pharmaceutical composition for oral administration, comprising the biotin moiety-conjugated polypeptide according to claim 1.

14. A pharmaceutical composition for oral administration for preventing or treating diabetes, comprising the biotin moiety-conjugated polypeptide according to claim 1.

15. A pharmaceutical composition for oral administration for preventing or treating obesity, comprising the biotin moiety-conjugated polypeptide according to claim 1.

16. A pharmaceutical composition for oral administration for preventing or treating fatty liver disease, comprising the biotin moiety-conjugated polypeptide according to claim 1.

17. The pharmaceutical composition for oral administration for preventing or treating fatty liver disease of claim 16, wherein the fatty liver disease is selected from the group consisting of simple fatty liver disease, non-alcoholic fatty liver disease, nutritional fatty liver disease, starvation fatty liver disease, obesity fatty liver disease, diabetic fatty liver disease, steatohepatitis, liver fibrosis, liver sclerosis and cirrhosis.

18. A pharmaceutical composition for oral administration for preventing or treating irritable bowel syndrome, comprising the biotin moiety-conjugated polypeptide according to claim 1.

19. A pharmaceutical composition for oral administration for preventing or treating neurodegenerative disease, comprising the biotin moiety-conjugated polypeptide according to claim 1.

20. The pharmaceutical composition for oral administration for preventing or treating neurodegenerative disease of claim 19, wherein the neurodegenerative disease is selected from the group consisting of Alzheimer's disease, Parkinson's disease, progressive supranuclear palsy (PSP), multiple system atrophy (MSA), Lewy body dementia, Parkinson's disease dementia epilepsy, stroke, Huntington's chorea, cerebral hypoxia, multiple sclerosis, and peripheral neuropathy.
